(19) United States
(12) Patent Application Publication
(10) Pub. No.: US 2005/0267450 A1
(43) Pub. Date: Dec. 1, 2005

(54) Ophthalmic treatment apparatus

(75) Inventors: Yuko Harumoto, Tokyo (JP); Hiroshi Koizumi, Tokyo (JP); Masayuki Momiuchi, Tokyo (JP); Wataru Oyagi, Tokyo (JP)

Correspondence Address: FOLEY AND LARDNER;SUITE 500, 3000 K STREET NW, WASHINGTON, DC, 20007, US

(73) Assignee: KABUSHIKI KAISHA TOPCON

(21) Appl. No.: 11/085,239

(22) Filed: Mar. 22, 2005

(30) Foreign Application Priority Data
May 26, 2004 (JP) 2004-156093

(51) Int. Cl. A61B 18/20

(52) U.S. Cl. 606/4

(57) ABSTRACT

An ophthalmic treatment apparatus has a main body and a light source unit. The main body has illumination means for illuminating illumination light to an eye to be examined, and observation means for observing reflected light from the eye to be examined. The light source unit has a light source for generating treatment beam having a predetermined wavelength, light-guiding means for guiding the treatment beam generated from the light source to a desired region of the eye to be examined and for irradiating the eye, and a casing. The light source and the light-guiding means are integrally built in the casing. The treatment beam is made incident from the light source directly to the light-guiding means.

### BACKGROUND OF THE INVENTION

1. Field of the Invention

The present invention relates to an ophthalmic treatment apparatus.

2. Related Art

Conventionally, an ophthalmic treatment apparatus for performing photocoagulation treatment by irradiating irradiation beam on an eye to be examined has been known. Since the irradiation beam used in such a laser beam treatment apparatus is used for a human body such as the inside of an eye, predetermined conditions for the security of human body are established. For example, in the case of irradiating a spot on fundus, it is necessary that the energy density of irradiation beam when the beam passes through cornea be a low value at which affect to cornea is little.

Further, such an ophthalmic treatment apparatus generally has light source means for irradiating laser beam as the irradiation beam and a light-guiding optical system (light-guiding means) for guiding irradiated laser beam, and the light source means and the light-guiding means are generally provided in separate bodies via optical fiber.

Japanese Patent No. 2018046, Japanese Examined Patent Publication No. 6-91892, and Japanese Unexamined Patent Publication No. 2002-253598 disclose such a conventional ophthalmic treatment apparatus.

However, since the light source means and the light-guiding means of the conventional ophthalmic treatment apparatus are provided in separate manner, total transmission efficiency reduces, if they are connected by optical fiber, for example, as follows:
Transmission efficiency of optical fiber: 70 to 80%
Transmission efficiency of slit lamp: 70 to 80%
→Total transmission efficiency: 50 to 60%

Therefore, it is necessary to use a light source of high output taking loss into account in order to bring the transmission efficiency of irradiation beam to a desired value.

On the other hand, bouncing by iris occurs when luminous flux is made thicker in order to reduce the energy density.

Accordingly, the range of NA value is inevitably determined when an irradiation spot diameter is previously determined on specification. Particularly, in the case of using optical fiber, a spot diameter becomes larger than the core diameter of optical fiber, and thus an irradiation optical system becomes a magnification system. This leads to strict NA conditions, and it is more likely to eliminate most of the level of freedom in designing the optical system.

### SUMMARY OF THE INVENTION

It is an object of the present invention to provide an ophthalmic treatment apparatus whose level of freedom in designing an optical system is increased and which is capable of realizing higher optical performance.

According to the present invention, an ophthalmic treatment apparatus has a main body and a light source unit that is freely detachable from the main body.

The main body has illumination means for illuminating illumination light onto an eye to be examined, and observation means for observing reflected light from the eye to be examined. The light source unit has light source means for generating treatment beam having a predetermined wavelength, light-guiding means for guiding the treatment beam generated from the light source means to a desired region of the eye to be examined, and a case.

The light source means and the light-guiding means are integrally built in the housing, casing or case.

It is preferable that the light source unit is freely detachably from the ophthalmic treatment apparatus main body in the state where the optical axis of the optical system is aligned with respect to the main body. Particularly, it is preferable that the junction portion between the light source unit and the ophthalmic treatment apparatus main body be formed in a bayonet type and freely detachable. For example, it is preferable that the light source unit and the ophthalmic treatment apparatus main body are joined by providing protrusions and holes that engage with the protrusion between them.

Another detachable mechanism can be employed in the junction portion between the light source unit and the ophthalmic treatment apparatus main body.

It is preferable that the light source means be constituted by a semiconductor pumped solid-state laser.

Further, it is preferable that the light-guiding means has a lens group and the treatment beam generated from the light source means is directly irradiated on the lens group.

Furthermore, it is preferable that the ophthalmic treatment apparatus main body have a mirror barrel and the end portion of the case or casing be freely detachable to the side surface area of the mirror barrel.

According to the present invention, the level of freedom in designing the optical system can be increased and higher optical performance can be realized.

The present invention has the following advantageous effects in particular:

(A) Optical Effects When Optical Fiber is Not Used

(1) According to the present invention, the transmission efficiency can be increased to a higher level. Particularly, the transmission efficiency of irradiation beam can be increased to 70 to 80%. Loss caused by optical fiber that has been conventionally used for connection can be eliminated. On the basis that power required on the spot is secured by high efficiency of the optical system itself, a light source of lower output can be used.
(2) Since there are cases where lifetime of a light source is drastically shortened when it is used at its maximum output state, the lifetime of the light source can be made longer when the light source is used at a low output.
(3) Limits of an irradiation optical system and an objective system on specification, which are caused by the limit of optical characteristic of optical fiber, are virtually eliminated.
(4) The level of freedom in designing the irradiation optical system is increased and high magnification zoom can be realized.
   (B) Structural Effects When Optical Fiber is Not Used

Operationality and security can be improved. The advantageous effects are as follows.
(1) Due to no optical fiber, a user can concentrate on an affected area when operating the apparatus.
(2) A trouble that optical fiber touches a patient can be prevented.
(3) A trouble that optical fiber gets stuck on the user/patient can be prevented.
(4) A possibility of causing damage (breaking, end surface damage) of the optical fiber is eliminated.
(5) A danger of falling of the apparatus is reduced.
(6) The user needed to pay attention to the operationality, security, and the stability of the apparatus when the fiber was thick, but the user does not need to pay attention to them in the present invention.
(7) The user needed to be careful because the fiber became invisible when it was thin, but the user does not need to pay attention to it in the present invention.
(8) The optical system can be smaller and lighter weight.
(9) Handling of the ophthalmic treatment apparatus becomes easier.

Further, the following advantageous effects can be obtained in case the light source unit (light source means and light-guiding means) is freely detachable to the ophthalmic treatment apparatus main body.
(1) Selectivity of output in the light source is diversified. A large number of light source units having various types of light source means built in are previously prepared, and a light source unit having a wavelength suitable for each treatment can be selected.
(2) In such a case, a type of laser light source can be easily selected in combination with a most suitable irradiation system and changed, so that most suitable settings corresponding to the type (wavelength, output) of light source can be easily made without the need of labor such as adjustment, and the apparatus can exert high performance. Specifically, the apparatus always can exert the maximum performance corresponding to the wavelength of light source while eliminating the need of adjustment to the optical system due to different wavelengths.
(3) When the specifications of the light source or an indication corresponding to them (indication such as color corresponding to wavelength, size corresponding to output, shape corresponding to spot diameter) are attached on the outer surface of the light source unit, mistake that might be caused in changing units can be prevented, and safe treatment can be performed.
(4) When the junction portion is constituted by a bayonet type, the unit can be easily detached.

### BRIEF DESCRIPTION OF THE DRAWINGS

Preferred embodiments of the present invention will be described referring to the drawings, in which:

FIG. 1 shows an example of an optical system of an ophthalmic treatment apparatus according to the present invention;

FIG. 2 shows another example of the optical system of the ophthalmic treatment apparatus according to the present invention;

FIG. 3 shows a state where the light source unit of FIG. 1 is changed to another light source unit;

FIG. 4 shows a front view of the ophthalmic treatment apparatus of FIG. 1;

FIG. 5 shows a side view of the ophthalmic treatment apparatus of FIG. 1;

FIG. 6 shows a front view of the ophthalmic treatment apparatus of FIG. 2; and

FIG. 7 shows a side view of the ophthalmic treatment apparatus of FIG. 2.

### PREFERRED EMBODIMENTS OF THE INVENTION

### Embodiment 1

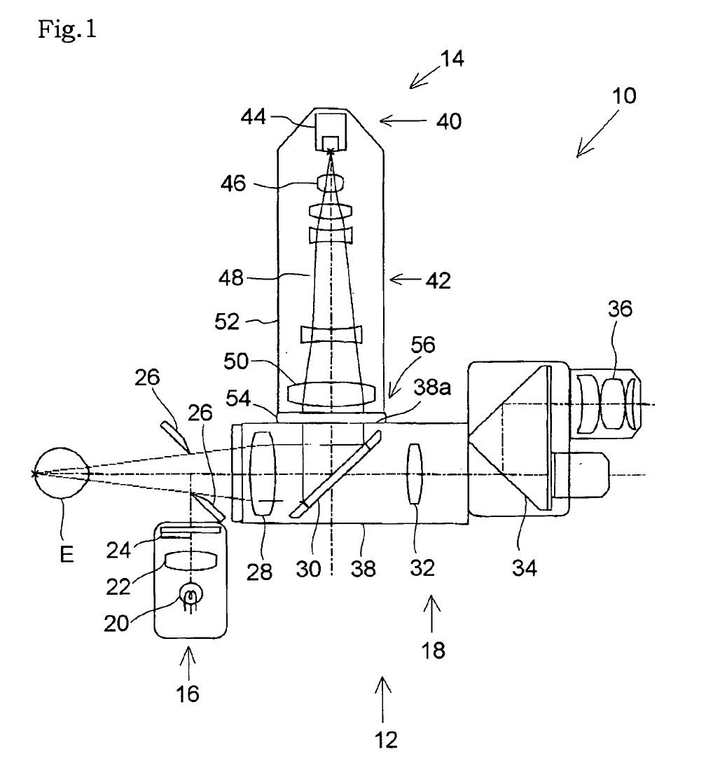

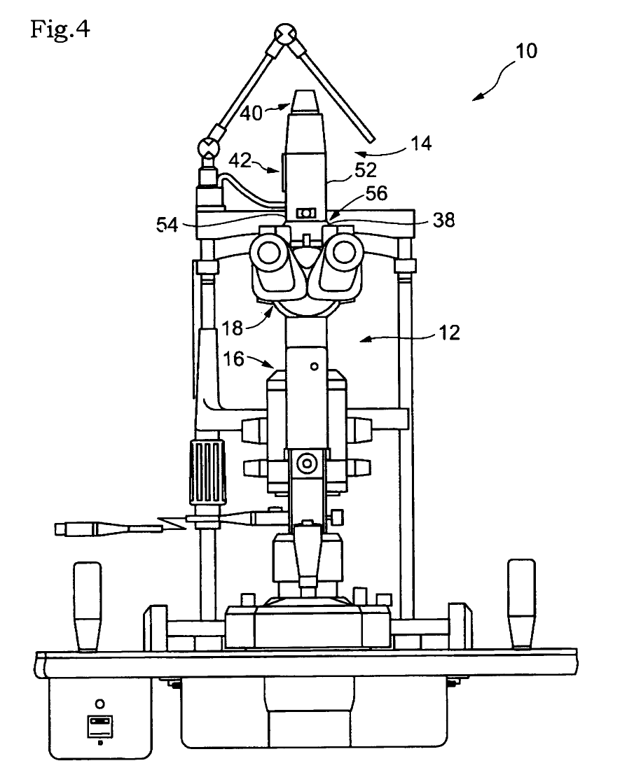

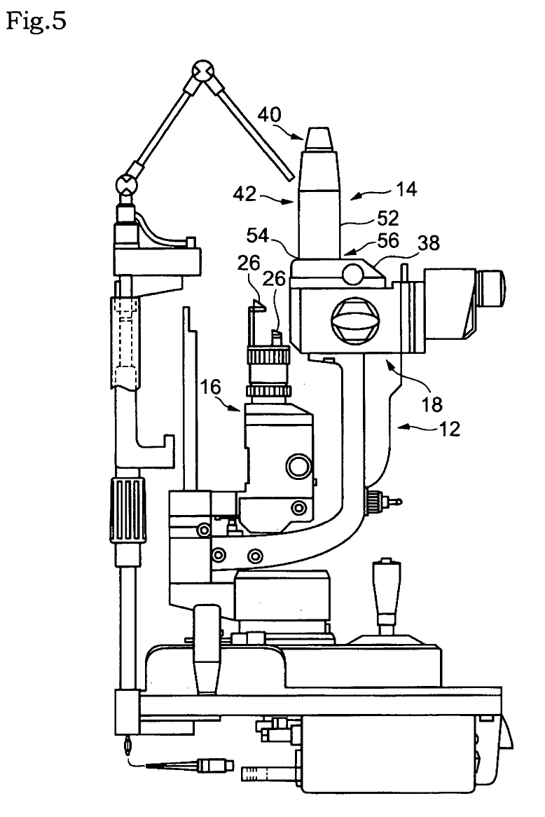

FIG. 1 shows an example of an optical system in an ophthalmic treatment apparatus according to the present invention. FIG. 4 shows the front view of the ophthalmic treatment apparatus of FIG. 1. FIG. 5 shows the side view of the ophthalmic treatment apparatus of FIG. 1.

An ophthalmic treatment apparatus 10 is an apparatus for performing laser treatment to the cornea or the like of an eye to be examined E. The ophthalmic treatment apparatus 10 has an ophthalmic treatment apparatus main body 12 and a light source unit 14 attached to the main body freely detachably.

The ophthalmic treatment apparatus main body 12 has illumination means 16 and observation means 18.

The illumination means 16 has a light source 20 such as a halogen lamp, a condenser lens 22, a slit 24, a reflective illumination mirror 26, and the like. The illumination means 16 is designed to reflect the illumination light illuminated from the light source 20 by the reflective illumination mirror 26 via the condenser lens 22 and the slit 24 and to illuminate the eye to be examined E.

The observation means 18 has an objective lens 28, a half mirror 30, a relay lens 32, a prism 34, an ocular 36, and the like. The observation means 18 is used to observe the image of the eye to be examined E by an examiner's eye (not shown). Note that the objective lens 28, the half mirror 30, and the relay lens 32 are housed in a mirror barrel 38. The light source unit 14 is to be attached to the upper surface area 38a of the mirror barrel 38.

The light source unit 14 has light source means 40, light-guiding means 42, and a case 52.

The light source means 40 has a laser light source 44. The laser light source 44 is made up of a semiconductor pumped solid-state laser, and irradiates irradiation beam having a predetermined wavelength.

Meanwhile, it is preferable that wirings (not shown) of power source and control for the laser light source 44 be set on positions where a patient or an operator does not touch them.

The light-guiding means 42 has a relay lens 46, a variable power optical system 48, and an objective lens 50. The relay lens 46, the variable power optical system 48, and the objective lens 50 constitute a lens group. The light-guiding means 42 guides the laser beam from the laser light source 44 to the eye to be examined E via the relay lens 46, the variable power optical system 48, and the objective lens 50.

The light source means 40 and the light-guiding means 42 are integrally built in the case 52. Therefore, the laser beam from the laser light source 44 can be directly guided and irradiated onto the eye to be examined E by the light-guiding means 42 without passing through optical fiber as in a conventional case.

An end portion 54 on the opposite side of the laser light source 44 in the case 52 and the upper surface area 38a of the mirror barrel 38 are constituted as a bayonet type junction portion 56. In other words, the light source unit 14 is constituted freely detachably from the ophthalmic treatment apparatus main body 12 in the state where the optical axis of the optical system is aligned with respect to the main body. A constitution is preferable in which the optical axis is aligned by providing protrusions and holes that engage with the protrusion between the end portion 54 and the upper surface area 38a.

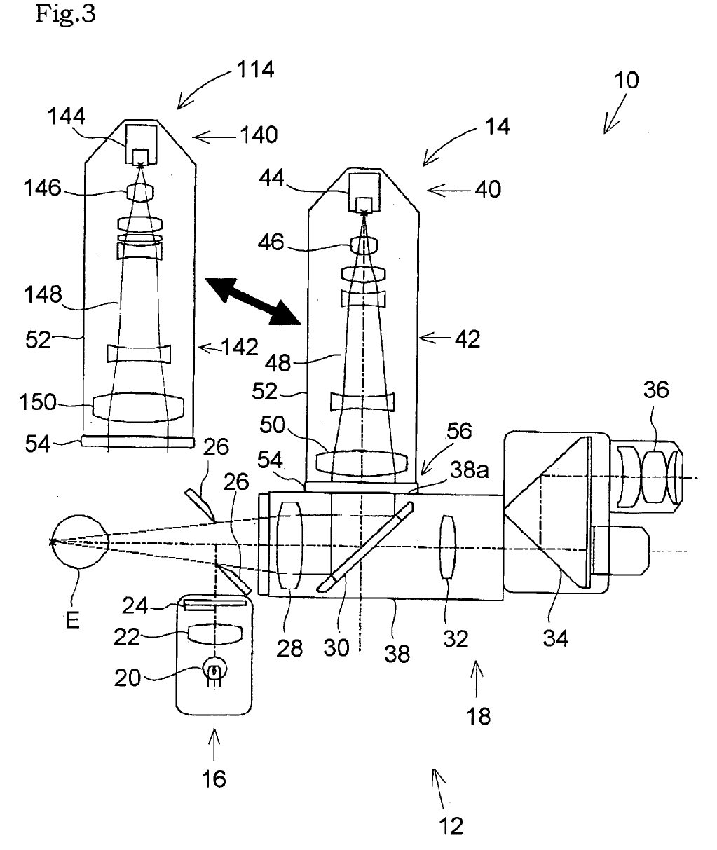

FIG. 3 shows a state where the light source unit 14 is changed to another light source unit 114.

Laser light source 144 of the light source unit 114 irradiates irradiation beam having a different wavelength from that of the laser light source 44 of the light source unit 14. The light source unit 114 is optimally set corresponding to the type (wavelength, output) of the laser light source 144, and can exert high performance without requiring labor such as adjustment.

Meanwhile, since light source means 140, light-guiding means 142, a relay lens 146, a variable power optical system 148, and an objective lens 150 are basically the same as the corresponding areas of the light source unit 14 except for the area described above, their explanation is omitted.

### Embodiment 2

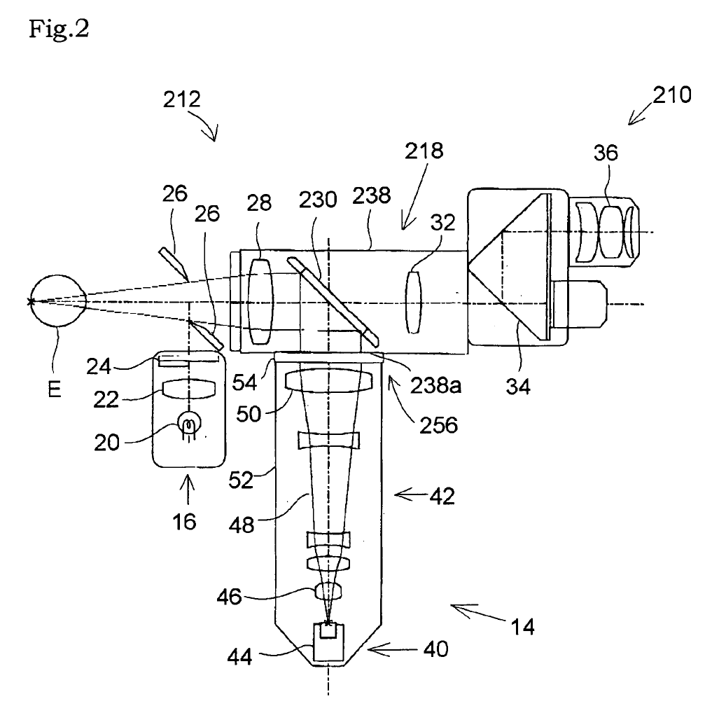

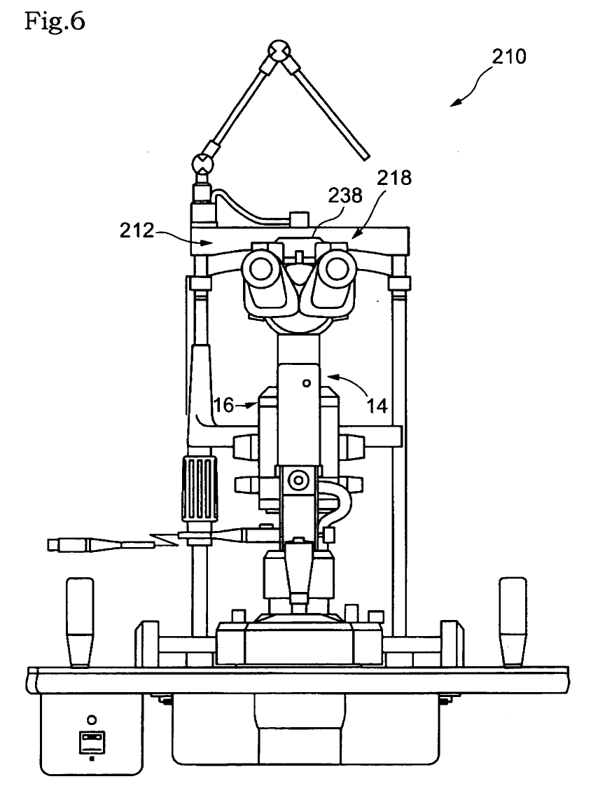

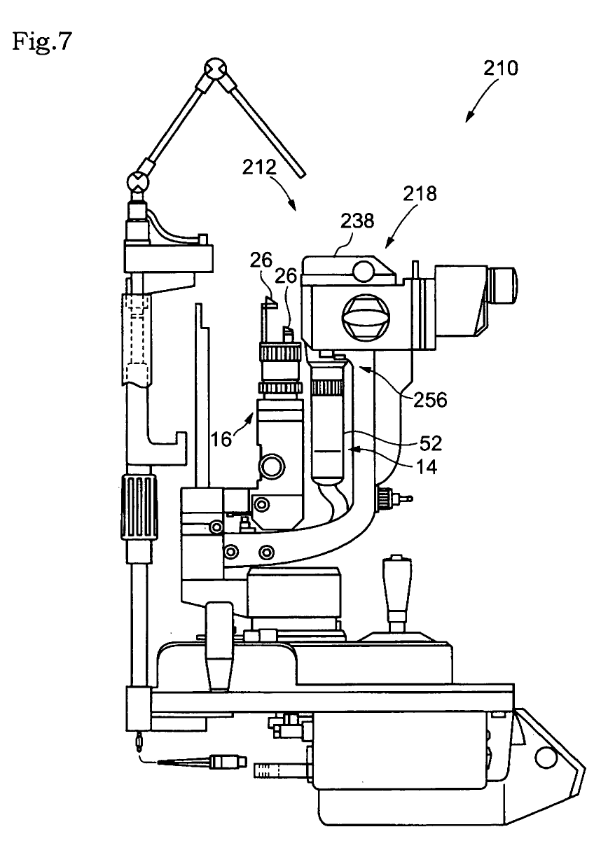

FIG. 2 shows another example of an optical system in an ophthalmic treatment apparatus according to the present invention. FIG. 6 shows the front view of the ophthalmic treatment apparatus of FIG. 2. FIG. 7 shows the side view of the ophthalmic treatment apparatus of FIG. 2. Note that reference numerals same as the above-described embodiment 1 are attached to the same members as the above-described first embodiment and their explanation is omitted.

An ophthalmic treatment apparatus 210 is an apparatus for performing laser treatment to the cornea or the like of the eye to be examined E. The ophthalmic treatment apparatus 210 has an ophthalmic treatment apparatus main body 212 and the light source unit 14 attached to the main body freely detachably.

The ophthalmic treatment apparatus main body 212 has the illumination means 16 and the observation means 18.

The observation means 218 has the objective lens 28, a half mirror 230, the relay lens 32, the prism 34, the ocular 36, and the like. The observation means 218 is used to observe the image of the eye to be examined E by the examiner's eye (not shown).

Note that the objective lens 28, the half mirror 230, and the relay lens 32 are housed in a mirror barrel 238. The light source unit 14 is designed to be attached to a lower surface area 238a of the mirror barrel 238.

The end portion 54 on the opposite side of the laser light source 44 in the case 52 and the lower surface area 238a of the mirror barrel 238 are constituted as a bayonet type junction portion 256. In other words, the light source unit 14 is constituted freely detachably from the ophthalmic treatment apparatus main body 212 in the state where the optical axis of the optical system is aligned with respect to the main body. A constitution is preferable in which the optical axis is aligned by providing protrusions and holes that engage with the protrusion between the end portion 54 and the lower surface area 238a.

The present invention is not limited to the above-described embodiments.

1. An ophthalmic treatment apparatus, comprising:
(A) a main body (12, 212), and a light source unit (14, 114) detachably attached to said main body (12,212),
(B) said main body (12, 212) having:
(b-1) illumination means (16) for illuminating illumination light to an eye to be examined, and
(b-2) observation means (18, 218) for observing reflected light from said eye to be examined,
(C) said light source unit (14, 114) having:
(c-1) light source means (40, 140) for generating treatment beam having a predetermined wavelength,
(c-2) light-guiding means (42, 142) for guiding the treatment beam generated from said light source means (40, 140) to a desired region of said eye to be examined (E), and
(c-3) a casing (52) in which said light source means (40, 140) and said light-guiding means (42, 142) are provided, and
(D) the treatment beam being introduced from said light source means (40, 140) directly to said light-guiding means (42, 142), in said casing (52).

2. The ophthalmic treatment apparatus according to claim 1, wherein said light source unit (14, 114) is freely detachable to said main body (12, 212).

3. The ophthalmic treatment apparatus according to claim 2, wherein a junction portion (56, 256) between said light source unit (14, 114) and said main body (12, 212) is formed in a bayonet type to be freely detachable.

4. The ophthalmic treatment apparatus according to claim 1, wherein said light source means (40, 140) is constituted by a semiconductor pumped solid-state laser.

5. The ophthalmic treatment apparatus according to claim 1, wherein said light-guiding means (42, 142) has a lens group (46, 48, 50), and the treatment beam is generated from the light source means (40, 140) and introduced directly to the lens group (46, 48, 50).

6. The ophthalmic treatment apparatus according to claim 2, wherein said main body (12, 212) has a mirror barrel (38, 238), and the end portion (54) of said casing (52) is freely detachable to a side surface portion (38a, 238a) of said mirror barrel (38, 238).
